(19) United States
(12) Patent Application Publication
(10) Pub. No.: US 2006/0286623 A1
(43) Pub. Date: Dec. 21, 2006

(54) Three dimensional object manipulating method and device

(76) Inventor: Hiroyuki Yonekawa, Dix Hills, NY (US)

Correspondence Address: Olympus America Inc/SEG, 3500 Corporate PKW, Center valley, PA, 18034-0610, US

(21) Appl. No.: 11/438,028

(22) Filed: Aug. 21, 2006

Related U.S. Application Data
(60) Provisional application No. 60/690,724, filed on Jun. 16, 2005.

(51) Int. Cl. C12M 1/34; C12Q 1/24

(52) U.S. Cl. 435/30; 435/287.3; 435/285.1

(57) ABSTRACT

This invention is used for the three dimensional microanalysis and observation of small object especially under microscope. This invention is applied on to the biomedical field as cell or tissue observation and manipulation. Three dimensional observation and manipulation method and device are cited in claim 1 and 2. And further cell or tissue manipulating technique as microinjecting, patch clumping and suctioning of living material is cited in claim 3.

### CROSS-REFERENCE TO RELATED APPLICATIONS

1. Science, 2004, Vol 305, August 13. P 1007-1009. “Optical Sectioning Deep Inside Live Embryos by selective Plane Illumination Microscope” Jan Huisken, et.al.
2. U.S. Pat. No. 5,338,997,
3. U.S. Pat. No. 6,275,335

Purpose of this invention: Current three dimensional observing method has several drawbacks as the need of complex and expensive equipment as Conforcal microscope, laser trap, mechanical manipulators or rotating stage. And they need longer operation time and tedious analysis steps. But true three-dimensional morphological analysis is requested and contributes the understanding of real three-dimensional structure of cell or tissue at biomedical research. This invention offers a convenient and cost effective three-dimensional analysis method of particle, cell or tissue using conventional microscopic stage and simple manipulating device. This three dimensional observation method and device can be applied to the particle analysis, environmental analysis, food industry and material science too.

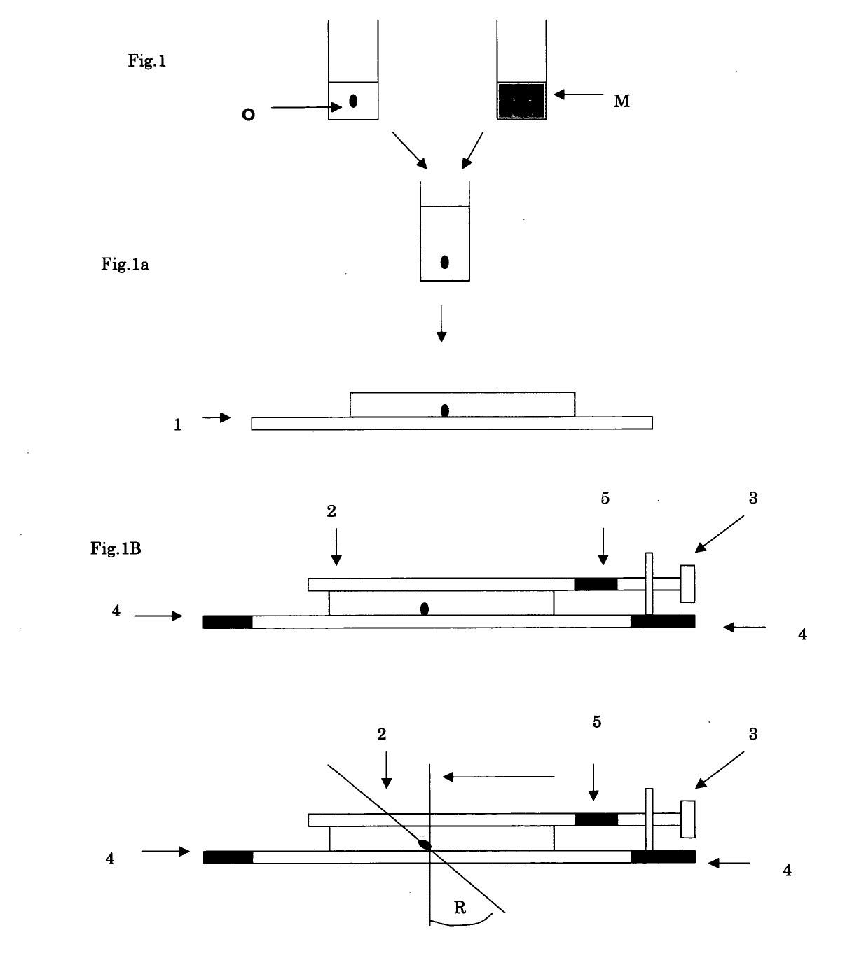

Embodiment 1, observation method. FIGS. 1a, b and c: A solution that contains a target object (O) is mixed with the viscous material (M), which is soft gel as agar, gelatin, polymers or matrigel. Then the mixture is placed on the transparent base plate as slide glass (1) as FIG. 1a. Them the viscous material (M) is covered with transparent cover (2) as FIG. 1b. That transparent cover (2) connects to the moving parts (3) by the connecting material (5). And the moving parts (3) are connected to the base chamber (4). The object (O) moves according to the micro-environmental movement of the matrix (M) between the cover (2) and base plate (1). The rotating angle (R) of object (O) is controlled according to the movement of surrounding material between the cover (2) and base plate (1).

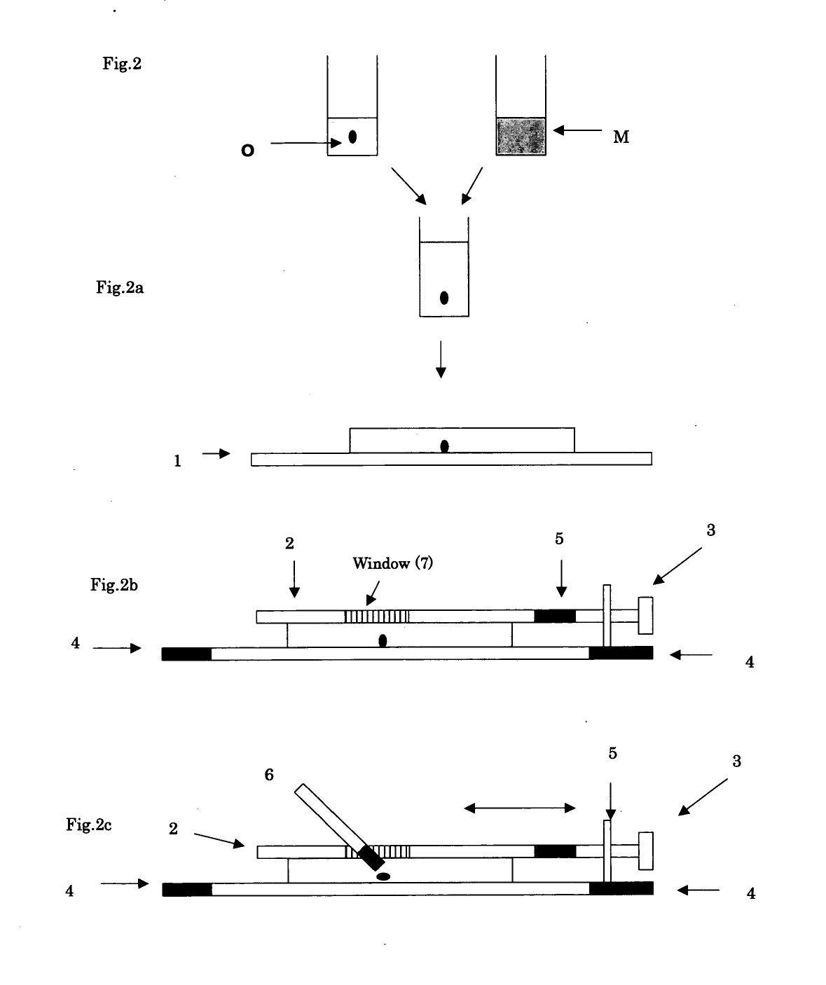

Embodiment 2. Microinjecting method (FIG. 2a, b and c): A solution that contains a target object (O) is mixed with the viscous material (M), which is soft gel as agar, gelatin, polymers or matrigel. Then the mixture is placed on the transparent base plate as slide glass (1) as FIG. 1a. Them the viscous material (M) is covered with transparent cover (2) with a hole (7) as FIG. 2b. That transparent cover (2) connects to the moving parts (3) by the connecting material (5) . And the moving parts (3) are connected to the base chamber (4) . The object (O) moves according to the micro-environmental movement of the matrix (M) between the cover (2) and base plate (1). At the optimal angle of object against the micro-injector (6), operator can operate a microinjection by micro-injector (6)

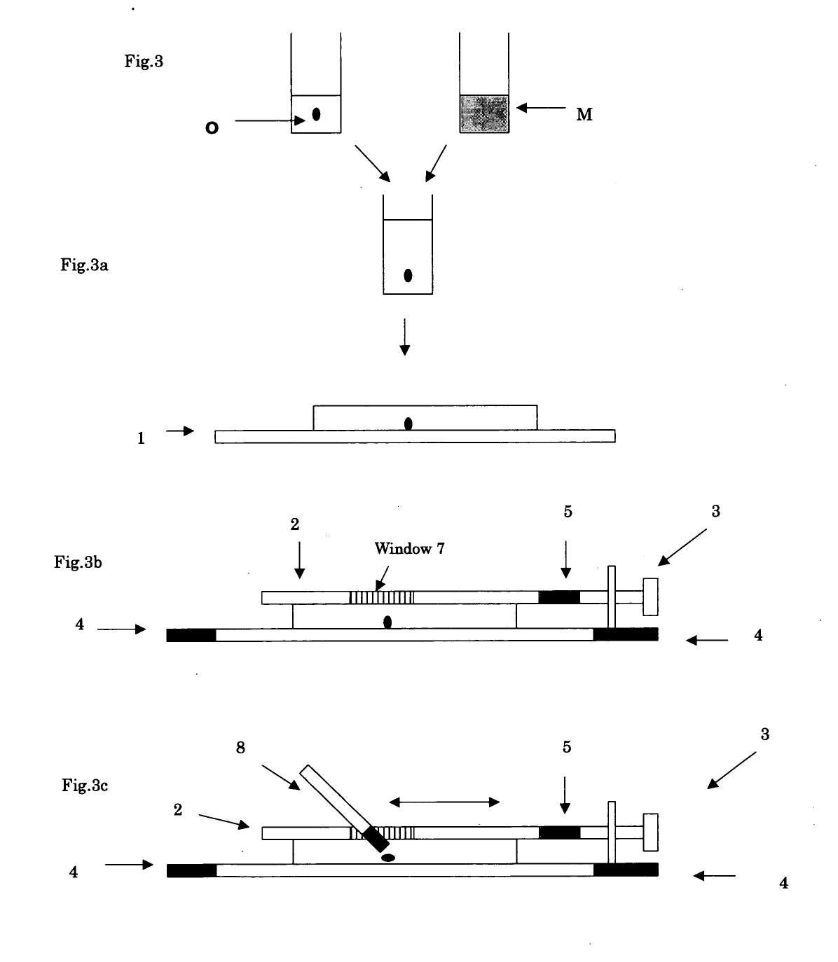

Embodiment 3 cellular components suctioning method FIG. 3a, b, and c): A solution which contains a target object (O) is mixed with the viscous material (M) which is soft gel as agar, gelatin, polymers or matrigel. Then the mixture is placed on the transparent base plate as slide glass (1) as FIG. 1a. Them the viscous material (M) is covered with transparent cover (2) with a hole (7) as FIG. 3b. That transparent cover (2) connects to the moving parts (3) by the connecting material (5). And the moving parts (3) are connected to the base chamber (4). The object (O) moves according to the micro-environmental movement of the matrix (M) between the cover (2) and base plate (1). At the optimal angle of object against the micro-pipettor (8), operator can operate cellular component suctioning by micro-pipettor (8).

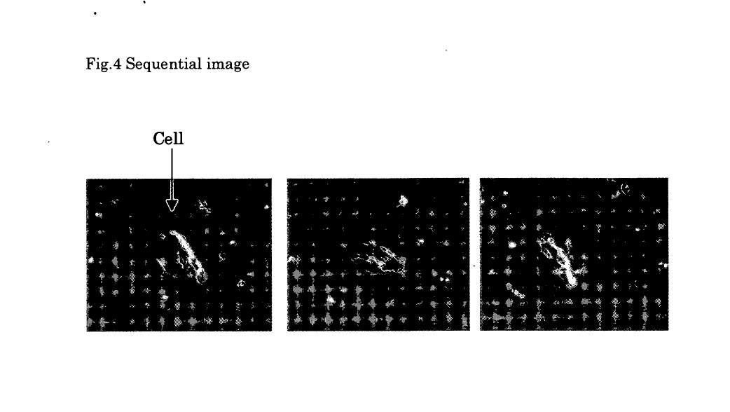

Embodiment 4: Actual three-dimensional observation of cell is shown in FIG. 4. Peripheral cells (C) are collected from human mouse and suspended in the phosphate buffered saline (PBS). The sampled cells are mixed with the 5% gelatin/PBS at heated condition as 40 degree C. After gently mixing cells in the test tube, that cell-gelatin mixture is placed onto the slide glass, then fixed in the observation chamber. Ordinal cover slip is placed on the sample. This cover slip was pushed by manual pipette tip by hand. The cellular image was taken by MIC-D microscope (Olympus America Inc) and use ×246 magnification. Taken images are saved and arranged by IBM PC T23.

### BACKGROUND OF INVENTION (PRIOR ART)

Three dimensional observation of cell and tissue are achieved by re-constructure from several sliced images by the conforcal microscopic analysis, mechanical micromanipulation, laser trap or rotation stage as cited Ref 1, 2 and 3. But re-constructuring 3D image from several sliced images is not actual image and just has fluorescence images only. And it takes longer processing time and not real time analysis. At the method of laser manipulator and mechanical manipulator, researcher can handle just single object at each operation. So researcher cannot observe multiple objects simultaneously. Rotating stage method uses capillary tube for the sample chamber that has spherical surface and it causes high distortion of image of object. And also researcher can observe only one axis rotating images.

### BRIEF SUMMARY OF THE INVENTION

This invention is cited on the manipulating method and device for the small object in a micro chamber. Small object as micro-particle, living cell or tissue fragment are mixed with a highly viscous medium as gel matrix or soft polymerized gel. Then the mixture transfer to the micro chamber which have transparent observing window and two-dimensional moving parts. After covering surface of the mixture by transparent cover, matrix embedded object can be manipulated by the movement of the transparent cover. The rotating angle of the embedded object is controlled arbitrary by the weak shear force between the transparent cover and transparent observing window. The invention provides simple three-dimensional observation method of small particle, cell or tissue under the microscope. Precise analysis of microstructure of small object as cell or tissue can be achieved. And this device provides further manipulation, dissection, extraction or micro-experiments of small particle, cell or tissue in correct angle.

1. Three dimensional object manipulating method by embedding object in the viscose material between two transparent plates at first, then move transparent cover or base plate and rotate embedded object in desired angle by the micro-movement of surrounding medium.

2. Three dimensional object manipulating device composed with two transparent plates, two axis micro-moving screw and connecting parts, which connect the screw and one of the transparent plates.

3. Microinjecting, patch clumping and suctioning of living material as cells and tissue by micropipette through the open area of the transparent plate of the said device by claim 2.
